(19) United States
(12) Patent Application Publication
(10) Pub. No.: US 2016/0183607 A1
(43) Pub. Date: Jun. 30, 2016

(54) Protective Clothing Article Including Fall Sensors and Deployable Air Bags

(71) Applicant: Biosensor LLC, Indianapolis, IN (US)

(72) Inventors: Alfredo Lopez Yunez, Indianapolis, IN (US); Diana Vasquez Torres, Westfield, IN (US); Ajay Kumar Bandi, Bloomfield Hills, MI (US)

(73) Assignee: Biosensor, LLC, Indianapolis, IN (US)

(21) Appl. No.: 14/619,908

(22) Filed: Feb. 11, 2015

Related U.S. Application Data
(60) Provisional application No. 61/938,138, filed on Feb. 11, 2014.

(51) Int. Cl. A41D 13/018; A41D 1/00; A61F 5/02

(52) U.S. Cl. CPC A41D 13/018; A61F 5/028; A61F 5/026; A41D 1/002

(57) ABSTRACT

A protective clothing article including a wearable member placeable over the torso of a user, includes an upper portion for engaging the user's shoulders, and a lower portion disposed, when worn by the user, at a vertical position generally similar to the pelvis of the user. At least two deployable airbags are disposed on the lower portion of the clothing article at a vertical position generally similar to the pelvis of a user. A compressed air source is provided for injecting air into the air bags upon deployment of the air bags to inflate the airbags. The clothing article also includes at least two sensors capable of detecting and sensing information relating to the direction and velocity of movement of the user. A controller is in communication with the sensor for processing sensed information from the sensor and processing said sensed information to determine whether a fall event is imminent and, upon determining that such a fall event is imminent, sending a signal to the compressed air source to inflate and thereby deploy at least one of the two deployable airbags.

### PRIORITY CLAMS

This Provisional application is related to, and claims benefit to Alfredo Lopez Yunez et al U.S. Provisional Patent Application Ser. No. 61/938,138, that was filed on 11 Feb. 2014 which are fully and completely incorporated by reference herein.

### I. TECHNICAL FIELD OF THE INVENTION

The present invention relates to medically related safety devices, and more particularly of a device for protecting persons against injuries caused by falling

### II. BACKGROUND

The fall is a very risky factor in elderly people's daily living, especially the independently living elderly. Due to their reduced recuperative powers, greater propensity to become injured, and often limited mobility, a fall occurring to the elderly often cause serious physiological injuries, such as bleeding, fracture, and central nervous system damages. If emergency treatments are not performed in a timely manner, these injuries may result in disability, paralysis or even death. On the other hand, fall may produce psychological problems such as fear of movement, and worry about living independently. It is estimated that over one third of adults of ages 60 years and older fall each year, making it a leading cause of nonfatal injury for that age group.

In 2002, about 22% of community-dwelling seniors reported falling events. The average Medicare costs of these fall events averaged between $9,113 and $13,507 per fall. In 2000, it is estimated that falls among older adults cost the U.S. health care system over $19 billion and that the cost of falls to the elderly cost the US healthcare system about $30 billion in 2010. With the population aging, both the number of falls and the costs to treat fall injuries are likely to increase. By 2020, the annual direct and indirect costs of fall injuries are expected to reach $54.9 billion.

It is estimated that about one in three adults aged 65 and older is subjected to a fall event each year. Of those, 20% to 30% suffer moderate to severe injuries that reduce the ability of the elderly person to live independently, thereby forcing them into living with others or at an institution such as an assisted care facility. These falls also increase their risk of early death.

Older adults are five times more likely to be hospitalized for fall-related injuries than injuries for any other cause. In 2009, about 20,400 older adults died from unintentional fall injuries. In the same year, emergency departments treated 2.4 million nonfatal injuries among older adults; more than 662,000 of those patients were hospitalized.

A variety of actions can be taken to reduce the likelihood of falls occurring, and to reduce the severity of injuries caused by such falls. One such set of actions involves improving the environment in which the elderly person resides to make it less likely to induce fall in its occupant. Many falls happen in homes and are to some extent preventable. Simple changes in lighting, housekeeping and furniture arrangement can make older adults less susceptible to falling in their homes.

For example, all rooms in older adults' homes should be well-lit. Brighter light bulbs should be employed and lighting should be added to dark areas. Night lights should be installed in bedrooms, bathrooms and hallways.

Clutter and tripping hazards can cause a person of any age to fall. All pathways should be kept clear and clean. Furniture should be arranged to ensure that there is always a clear pathway to enter and exit a room. In high danger areas such as bathrooms, grab bars should be installed to give the person something to hold on to promote their stability.

Many falls occur on stairs and steps. All stairwells should be well-lit, clear of all objects and have handrails on both sides. Optimally, elderly people are much safer living in environments where they do not have to either climb or descend stairs as a part of their daily activity

Another method for reducing the severity of falls experienced by the elderly is to provide the elderly person with a cushioning system that cushions the impact of any fall on the user's body. For example one or more appropriately placed cushioning members, such as pads could be strategically placed around the user's body to help absorb the impact of a fall on those parts of the user's body most likely to be injured in a fall.

Conceptually, such padding members could be constructed and positioned similarly to the various pads that comprise components of modern hockey or football protective gear. Currently, such padded, injury reducing clothing products are available from a variety of manufacturers. An example of one such product is the AliMed® HipShield® Hip Protector

Another type of device for helping to reduce the impact of falls are a series of devices that comprise wearable products having air bags that are inflatable in the case of a fall to help cushion the user. Examples of such devices are shown in Alstin et al., U.S. Pat. No. 8,402,568; Buckman, U.S. Pat. No. 7,017,195; Buckman, U.S. Pat. No. 7,150,048; Ishikawa et al., U.S. Pat. No. 7,548,168 and January, U.S. Pat. No. 8,365,416.

One of the difficulties with employing a selectively actuable-type airbag system is designing a system that is capable of accurately detecting fall events, so that the airbags are deployed at an appropriate time. As deployment of the airbag may end the useful life of the product, one would not wish to deploy an airbag if a fall is not about to occur, since that would extinguish the airbag's useful life, along with making the device cumbersome to the user. As such, it is important to be able to provide a sensor that will avoid such false positives.

Similarly, false negatives can be just as problematic, as the failure of a sensor to detect a fall event when it is occurring, can cause an airbag to fail to deploy. The failure of an airbag to deploy during a fall event prevents the device from performing its intended function and serving its intended purpose of cushioning the user's fall.

It will be also appreciated that a wearable device having a deployable cushion is not going to be held statically. Rather, the device will move in conjunction with the movements of the user. Because of the complexity of the movements and the different types of movements, difficulties arise in distinguishing between fall events where the airbags should be deployed, and non-fall events when the airbag should not be deployed.

Sensor systems are known to the Applicants that have attempted to appropriately distinguish between fall events and non-fall events. Examples of these sensors are discussed in the Bianchi et al., Nguyen et al., and Sposaro references set forth below.
F. Bianchi, S. Redmond, M. Narayanan, S. Cerutti, and N. Lovell, “Barometric pressure and triaxial accelerometry-based falls event detection,” Neural Systems and Rehabilitation Engineering, IEEE Transactions on, vol. 18, no. 6, pp. 619-627, December;
T.-T. Nguyen, M.-C. Cho, and T. S. Lee, “Automatic fall detection using wearable biomedical signal measurement terminal,” in Engineering in Medicine and Biology Society, 2009. EMBC 2009. Annual International Conference of the IEEE pp. 5203-5206, September; and
F. Sposaro and G. Tyson, “ifall: An android application for fall monitoring and response,” in Engineering in Medicine and Biology Society, 2009. EMBC 2009. Annual International Conference of the IEEE pp. 6119-6122, September

Although the sensor systems set forth above, and the devices disclosed in the patents mentioned above, likely perform their intended functions in a workmanlike manner, room for improvement exists

### III. SUMMARY OF THE INVENTION

In accordance with the present invention, a wearable apparel item is provided that provides cushioning members for lessening the impact of a fall of the wearer. The device includes a wearable member including a first portion that is disposed, when worn, adjacent to the hips of the user. A plurality of inflatable cushion members are disposed in the first portion. The cushion members include at least a first cushioning member that is disposed adjacent to user's first hip, and a second adjacent at a second cushioning member disposed to the user's second hip.

A sensor device is provided that is operably coupled to the cushioning members. The sensor member preferably includes an accelerometer and a processor. The processor includes an algorithm that is capable of distinguishing between falling events and non-falling events. The device includes an air inflation mechanism operatively coupled to the processor. Upon being actuated by the processor in the event of a fall event, the air inflation mechanism injects air into the formerly empty air cushions to cushion the impact of a fall on the user's hips.

In a preferred embodiment, the device comprises an inventive, data acquisition device comprising an accelerometer and the processor comprises a micro controller with an analog to digital converter

Most preferably, a communication device is capable of facilitating communications between the data acquisition unit and the processing unit, and between the processing unit and a remote data acquisition device, such as a computer or data receiving station.

### IV. BRIEF DESCRIPTION OF THE DRAWINGS

FIG. 1 is a front view of a person wearing a vest of the present invention;

FIG. 1A is a rear view of a person wearing the vest of the present invention;

FIG. 1.1 is a Block diagram of a typical sensor;

FIG. 2.1 is a schematic view to illustrate sensors placement on test subject;

FIG. 2.3 is a Front view of Proposed Industrial Lifting belt embodiment of the present invention;

FIG. 2.4 is a back view of Proposed Industrial Lifting belt embodiment of the present invention;

FIG. 3.1 is a schematic, block diagram of the integrated sensor system of the present invention;

FIG. 3.3 is a schematic, pin diagram of a ATmega328;

FIG. 3.4 is a schematic view illustration the system architecture of an MMA8452Q;

FIG. 3.5 is a schematic pin diagram of a MMA8452Q accelerometer;

FIG. 3.6 is a pin diagram of a PCA9544A IC2 bus multiplexer;

FIG. 3.7 is a schematic view of the PCA9544A I2C multiplexer showing its connections to other members of the circuit of the present invention;

FIG. 3.8 10 is a schematic view of the Circuit connections for the hardware prototype of the present invention

FIG. 3.9 is a schematic representation of a I2C timing diagram;

FIG. 3.10 is a front view of a user wearing an industrial lift belt type version of a fall detection unit of the present invention, to which the air bags had not yet been incorporated;

FIG. 3.11 is a front view of a user wearing an industrial lift belt type version of a fall detection unit of the present invention, to which the air bags had not yet been incorporated;

FIG. 4.1 is a schematic representation of a decision tree employed with the present invention;

FIG. 5.1 is a graphical representation of a Fall Detection Simulation (MATLAB Output Plot);

FIG. 5.2 is a graphical representation illustrating Accuracy Results by Number of Consecutive Samples used for Testing;

FIG. 5.3 is a graphical representation illustrating Detection Results Enrolling and Testing with same Data Set as the data of Data Set 2;

FIG. 5.4 is a graphical representation illustrating Detection Results Enrolling Data Set 2 and Testing on All Data (Data Set 1 and Data Set 2);

FIG. 5.5 is a graphical representation illustrating Total Simulation Detection Results Enrolling Data Set 2 and Testing on All Data 43;

FIG. 5.6 is a graphical representation illustrating Detection Results of Hardware Prototype for Falls only to illustrate the accuracy of the device in detecting fall results;

FIG. 5.7 is a graphical representation illustrating Detection Results of Hardware Prototype including No-Falls to illustrate the accuracy of the device in detecting fall results and no-fall results; and

FIG. 6.1 is a flow chart view of a Closed loop functioning diagram illustrating the decision flow of the present invention.

### V. DETAILED DESCRIPTION

### A. Discussion of the Vest

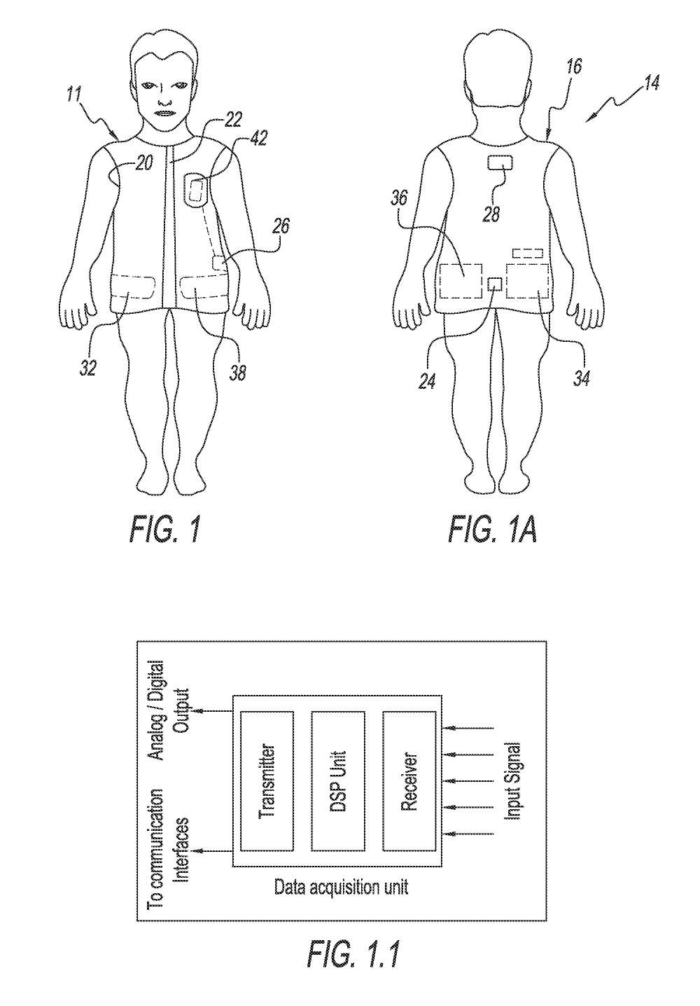

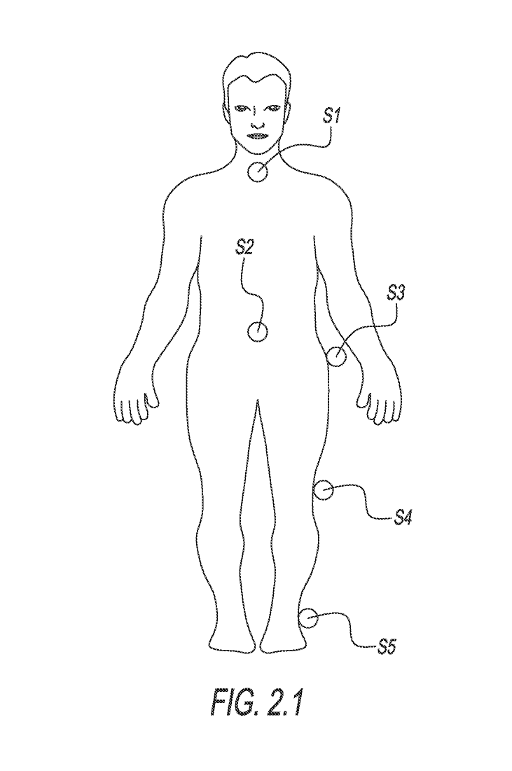

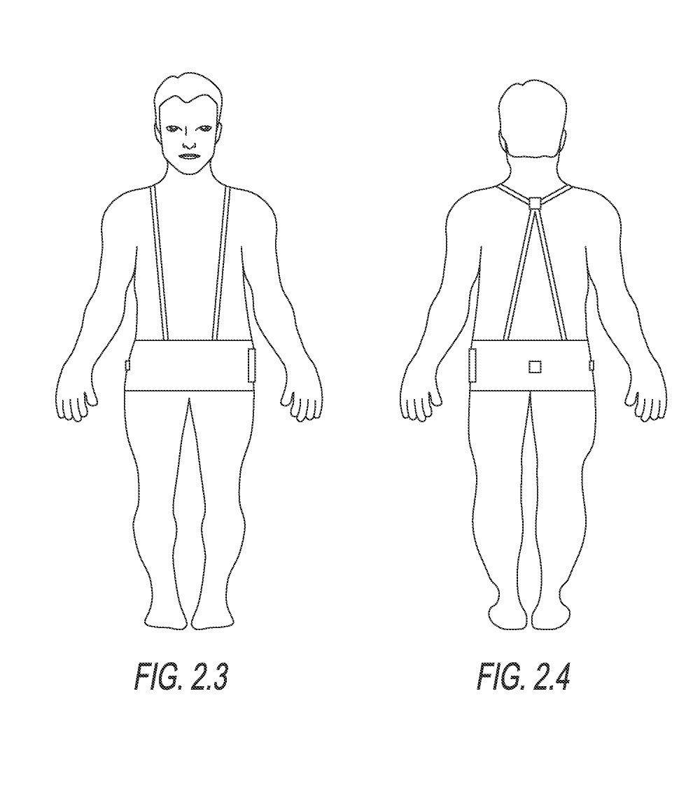

A vest that is useable with the present invention is shown in FIGS. 1 and 1A. The vest 14 includes a top edge that rests against the shoulder of the user and a lower edge that hangs generally below the hips of the user. Otherwise, the vest 14 generally fits over the torso of the user. A pair of arm holes 20 is formed to enable the user to extend his arms through the vest from the interior to the exterior.

The front closure member 22 extends down the front of the vest, and enables the vest to be opened, so that one may insert their torso therein. The front closure member 22 preferably comprises a pair of flaps having mating “book” and “eye” fasteners thereon, such as Velcro brand fasteners, so that the front closure member 22 can be moved between an opened position to enable the user to get in and out of the vest 14, and a closed position wherein the vest 14 will be maintained on the user.

In addition to Velcro closures, other closures such as buttons, snaps, or belts having buckles, such as plastic buckles of the type that one normally finds on life vests. Preferably, the vest 14 is made of a thin and lightweight fabric so as to reduce weight. In order to make the vest easily cleanable, the vest may be designed to be made of a nylon or Dacron material similar to a life preserving vest that can be easily cleaned by wiping the surface, and that is generally imperious to liquids and body fluids that may be spilled onto the vest, or food material that may be dropped onto the vest. Additionally, the material from which the vest 14 is made should have a pleasing appearance.

Because of the electronic components, such as the sensors, controller and the air canisters used with the device 10, and due to the long projected useful life of the vest 14, accommodations should be made so as to enable the user to clean the vest 14. These accommodations can be made through a variety of vehicles. For example, one could ruggedize and waterproof the electrical and other components, so that the vest can be placed in a washing machine and washed like clothing. Alternately, the electrical and other components that are likely to be damaged by washing may be designed to be selectively removable, so that such water damageable components can be removed before the vest is placed in a washing machine, and then re-inserted when the cleaning process is finished.

A second alternative is to make the vest of a material having a water and fluid impervious surface, such as Dacron, Rayon, vinyl and the like, so that the device can be cleaned by wiping the vest off with soap and water or other cleaning solution.

Preferably, the vest 14 includes three sensors, including a first sensor 24, a second sensor 26 and a third sensor 28. The first sensor 24 is preferably placed in the center of the back portion of the vest 14. The second sensor 26 is preferably placed near the bottom of the vest 14, and along the side. Sensor 26 is shown as being placed on the left side of the vest 14. The third sensor 28 is preferably placed in an upper portion of the vest 14, on the back thereof.

As will be described in more detail, the first, second and third sensors 24, 26 28 respectively, preferably comprise accelerometers. Each of the sensors 24, 26, 28 is in communication with a controller member 42 that is preferably placed in a position similar to the position of a “cigarette pocket” of a shirt or vest.

As will be described in more detail below, the controller 42 is in communication with both the sensors 24, 26, 28 and the airbags 32, 34, 36, 38 and airbag canister 44. The communications between the various components can be through a hard wired connection or else a wireless communication, such as a Bluetooth communication connection. As will also be described in more detail below, the controller 42 can be in communication with a remote device, such as a computer, central monitoring station or the like (not shown) to communicate information about the user to either a data storage device for storing the information, or some data monitoring type device, that can alert another person that the user has undergone a fall event, and may need attention because of the fall event.

The airbags include a first airbag 32, a second airbag 34, a third airbag 36 and a fourth airbag 38. The airbags 32, 34, 36, 38 are normally in a deflated configuration, and are movable between a deflated configuration and an inflated configuration through the insertion of a quantity of air into the air bags 32-38. When in the inflated position, the airbags 32-38 provide a cushioning that is designed to reduce the force of an impact, such as the force exerted by a floor on a user's hip, when the user falls. It will be noted that the airbags 32, 34, 36, 38 are positioned generally adjacent to, and at the same vertical level as the hips of the user.

The first airbag 32 is placed on the front, right hand surface of the user. If one were to assume that the front closure member 22 is placed at a “12:00” position on a user, the first airbag 32 would be placed at approximately 2:00. The second airbag 34 is placed at approximately “4:00” to protect the user's side and rear portion, in a manner similar to the manner in which the first airbag 32 protects the user's front and side hip portions. The third airbag 36 is placed on the rear of the vest 14, on the user's left side, and generally protects the user's rear and side hip portions. The fourth airbag 38 is placed on the front, adjacent to the first airbag 32, and is placed on the front left side of the user to protect the user's front and left side area.

An air canister 42 is provided for containing compressed air that can be ejected into the airbag 32-38 upon receipt of a signal from the controller that a fall event is occurring. In lieu of air, alternate gases such as carbon dioxide, oxygen, and helium can be employed. Such signals usually occur in response to a signal being given by the sensors that a fall event has been sensed by the sensors. This sensed fall is then processed by the controller that, as will be discussed below, is capable of distinguishing a real fall from a false positive fall, so that the airbag is only inflated upon a fall event.

Depending upon the sophistication of the computer, the canister 12 can be designed, through either the use of valving or multi-canisters, to inflate one, two, three or all four airbags. Depending upon the nature of the fall, it may be desirable to only inflate a pair or airbags, such as airbag 32 and 38 if the user is making a forward fall, or alternately, airbags 34, 36 if the user is falling backwardly.

In addition to airbags 32-38 protecting the hips, the vest 14 can be designed in another configuration, or have airbags disposed at other places to protect other potentially injurable parts of the user. However, it is currently believed that the injuries to the hips are the greatest concern.

The airbags used in connection with the present invention in theory, operate similarly to an airbag in an automobile. However, significant differences exist. In particular, because the severity of the impact and the speed of the fall is much less when a person is falling down, when compared to a car wreck, it is likely that the airbags 32-38 will not need to deploy as quickly, or as violently as airbags in an automobile.

The sodium azide airbags currently used in many automobiles produce highly satisfactory results since they can inflate an airbag within 60 to 80 milliseconds However, gases such as sodium azide have deleterious health effects, and can burn the user.

Other gases, such as compressed air and nitrogen will likely work well with the present invention due to the fact that the almost explosive inflation provided by the sodium azide gas used in automobiles is not necessary to protect the user in the case of a fall from a standing position or a fall from a chair. For example, currently existing airbag vests that are used in connection with the protection of motorcyclists employ a carbon dioxide cartridge. See for example the motorcycle vests shown at, www.Bikebone.com that are produced by Bike Bone.com of Union City, Ga.

One of the difficulties that faced the Applicants when designing a sensor system and control system for use in connection with the airbag vest of the present invention is to produce an accurate sensor system that will deploy an airbag if an actual fall occurs, but will not deploy an airbag if a movement occurs that is not a fall event.

As will be appreciated, the failure of the device to deploy an airbag in the event of a fall event would serve the user no good, as the undeployed airbag would not help protect the user from injury. On the other hand, a false deployment of an airbag in the event that no fall was occurring, might cause the useful life of the airbag to end, as the device may be designed for one airbag deployment use. Even if multiple deployments were possible with the particular vest, there would likely be time and energy expended in repacking the airbag and replacing the gas cartridge used to inflate it.

Discussed below is the design of the electronic componentry used in connection with the vest of the present invention, along with the discussion of the testing that was performed to ensure that the device performed as desired.

### B. The Components

### 1. The Sensors

### (a) Wireless Applications

A sensor is a device, which can convert physical information into signals, which can be interpreted by a user using an electronic component. Usually the signals received from these sensors are in analog form and can be converted and formatted into digital by using computers. With the advent of technology we can now use sensors, which are smart and efficient enough such that they come with all the processing and conversion units on the sensor body itself. These smart sensors are energy efficient and they also have embedded functions to communicate, transfer data and can also take inputs from the computers to accomplish the applications.

Smart sensors can be used to design integrated data acquisition systems, where they are used to obtain data continuously, process the data so acquired, and implement the data in their respective applications to accomplish the tasks assigned. High-resolution data is expected to be obtained from these sensors so that the uncompromised accuracies can be obtained. Sending these high-resolution data streams to remote computers in real-time gives the user or operator the ability to monitor and store the data efficiently. It also reduces the size of the processing unit, which is supposed to be with the person at all times and which is worn as a part of the vest, and is incorporated into the vest.

### (b) Embedded Sensor System Applications

Sensor technology has been used in measuring different physical quantities such as position, temperature, humidity, orientation, pressure, torque, radiation, acceleration and many more. With this wide range of capabilities, sensors find their applications in many areas in our day-to-day life. Applications include Medical, automotive, industrial, HVAC (heating, ventilation, and air conditioning), civilian etc. In this application, the primary areas of concern are medical and civilian usage of sensors to monitor and protect elders from fatal fall occurrences in real-time.

### (c) Sensor Fabrication Techniques

Semiconductors play a major role in sensor manufacturing using advanced techniques like MEMS (Micro-Electro Mechanical System), lab-on-chip, system-on-chip and ASIC (Application-Specific Integrated Circuit). These sensors are capable of doing data acquisition and signal processing at the same time consuming the lowest possible power. FIG. 1.1 explains the basic digital processing system inside a typical sensor. Initially, the physical data is obtained from the sensing area and the receiver section turns it into the digital signal-processing unit. Here the analog signals are converted to digital signals using A/D converters. The output of this block is then given to the transmitter section, where the data can be transmitted to other circuits like microprocessors or computers using various communication protocols, some of them include I2C, SPI and UART. A block diagram of a typical sensor is shown in FIG. 1.1.

In the present invention accelerometers are used as the sensor units, which can record the patients' physical activity. There are different types of accelerometers and what differentiates them is the type of sensing element and the principle of operation involved. The following is the list of typical accelerometers in use

Capacitive:

Capacitive accelerometers sense the change in the electrical capacitance between static condition and dynamic state with respect to acceleration.

Piezoelectric:

Piezoelectric accelerometers use materials such as crystals, which generate electric potential from an applied stress, also called as the piezoelectric effect.

Piezoresistive:

Piezoresistive accelerometers (strain gauge accelerometers) work by measuring the electrical resistance of a material when mechanical stress is applied.

Hall Effect:

Hall Effect accelerometers measure voltage variations stemming from a change in the magnetic field around the accelerometer.

Magnetoresistive:

Magnetoresistive accelerometers work by measuring changes in resistance due to a magnetic field. The structure and function is similar to a Hall Effect accelerometer except that instead of measuring voltage, magnetoresistive accelerometers measures resistance.

MEMS-Based Accelerometers:

MEMS technology is based on a number of tools and methodologies, that are used to form small structures with dimensions in the micrometer scale. The same technology is being utilized to manufacture state of the art MEMS-Based Accelerometers.

From industry to education, accelerometers have numerous applications. These applications range from triggering airbag deployments to the monitoring of nuclear reactors. There is a number of practical applications for accelerometers that are used to measure static acceleration (gravity), tilt of an object, dynamic acceleration, shock to an object, velocity, orientation and the vibration of an object.

In the present invention, the Applicants have found that the most preferred accelerometer is a MMA8452Q accelerometer. The applicants chose the MMA8452Q accelerometer for the following reasons.
1. It supports I2C communication (between accelerometer and the microcontroller).
2. It has two programmable interrupt pins for six interrupt sources:
   a. It provides flexible output data that can be configured to be in either 8-bit or 12-bit
   b. Motion/freefall detection is based on the configured threshold
   c. It can detect both Single and double taps.
   d. It has the ability to detect the orientation in all 6 orientations.
   e. It has a built in high-pass filter along with user configurable cut off frequencies, which features transient detection; and
   f. It has a built in auto-wake/sleep mode
3. It features dynamically selectable acceleration ranges of: ±2 g/±4 g/±8 g
4. Its output data rates can be chosen from 1.56 Hz to 800 Hz depending on the signal resolution required by the application

### (d) Communication Protocol

The digital data from the sensors is usually provided in the serial form. The present invention employs communication protocols that are intended to use in these sensor applications, where the data can be of high resolution and frequencies, ranging from KHz to few MHz. Some of them are Inter Integrated Circuit protocol (I2C), Serial Peripheral Interface (SPI), Universal Asynchronous Receiver and Transmitter (UART), and Universal Serial Bus (USB) [13].

In the present invention, I2C communication protocol is used, which is dependent on the clock frequency. All the communication and data transfer in this protocol is done with reference to the clock line. This I2C protocol uses bidirectional open drain lines, Serial Data line (SDA) and Serial Clock line (SCL) pulled up with resistors. The typical voltages involved in this communication are 3.3V or 5V.

The device that is controlling the other peripheral devices is called “master” and the devices connected to the master are called “slaves”. Each slave has its own address so that they can be invoked uniquely during operation. SPI is also similar to I2C but it has a chip select line to control the slaves connected to it. UART and USB communications are asynchronous communication protocols, where the data transfer and communication is done without a clock signal.

### (e) Approach Taken in the Present Invention

The present invention employs a tri-axial signal based on the fact that scientific data features like SVM, SMA, and tilt angle are calculated and fed into the decision tree algorithm to obtain the real fall thresholds while eliminating false falls. Threshold values, determined from experimental verification, are used in the fall detection prototype, and whenever a fall is detected, an LED is turned on, and the event is logged. This approach features high speed-low power from the use of low power and high speed embedded system processor. The algorithms used here also feature high speed to reach the processor decision in a timely manner.

### 2. Neurosciences and Neuro-Signals

Prior to designing a reliable and effective fall detection system, it is necessary to study the neurological inputs and pathways of balance and natural fall prevention in humans. People monitor their environment by constantly adjusting their orientation with respect to movements. Two particular systems use external inputs to perform this task and anticipate the occurrence of a fall: vestibular system and somatosensory system

### (a) Vestibular System

The vestibular system is in charge of engaging neurological pathways to provide perceptions of gravity and movement. The inner ear consists of a series of components that help transduce signals into electrical events. The membranes in the inner ear consist of three semicircular ducts (horizontal, anterior, and posterior), two otolith organs (saccule and utricle) and the cochlea, which is part of the auditory system. The semicircular ducts respond mainly to angular acceleration.

A head turning movement induces movement of inner fluids that bend the cilia of hair cells. This causes the external input to convert into neurosignals. The otolith organs are located against the walls of the inner ear and they also influence the transmission of signals during head movements through the VIII th nerve to the brainstem. The utricle organ has higher sensitivity when the head is upright, while the saccule is most sensitive when the head is in a horizontal position.

### (b) Somatosensory System

Somatosensory systems allow identifying the environment using physical touch. For instance, Somatosensory systems help to process information about characteristics of the environment such as temperature and pain through neural stimulation. Also, the somatosensory system of proprioception causes awareness of body position through muscle and joint stimulation. This sensory information is transported and processed by somatosensory systems along various pathways based on the type of information that is being transported. For particular muscle contraction or proprioceptive information is carried along the column-medial lemniscal pathway

### (c) Neurosignals

Different studies have shown how the proprioceptive system and muscle reactions influence body anticipation to a free fall in elderly subjects. Electromyography (EMG), a technique that helps study the muscle electrical activity, has helped to evaluate muscle activity during a fall. In Bisdorff's “EMG responses to free fall in elderly subjects and akinetic rigid patients”, EMG recordings in two normal subjects in response to randomly presented startling stimulus (fall) or non-startling stimulus (click) were analyzed. A. Bisdor, A. Bronstein, C. Wolsley, M. Gresty, A. Davies, and A. Young, Emg responses to free fall in elderly subjects and akinetic rigid patients,” J Neurol Neurosurg Psychiatry, vol. 66, no. 4, pp. 447-55, 1999.

The subject's task was to dorsiflex the ankles in response to either stimulus. The fall induced startle occurred at about 100 ms followed by the voluntary contraction at about 200 ms. To assess the relative strength of the response, the rectified EMG areas were normalized in individual subjects by setting the strongest single activation found at an arbitrary level of 100%.

The mean EMG strength was significantly larger in response to the startling stimulus (fall=78.6 (SD 17.2)) than to the non-startling stimulus (click=50.4 (SD 18.5); arbitrary % EMG units; p=0.0001). It was concluded that in the case of a free fall it seems reasonable to assume that, in normal subjects, the vestibular system is important. However, as the data suggest, patients with a longstanding absence of vestibular function are capable of using other sensory sources to generate the response. Contact and proprioceptive signals, particularly from the neck, have access to the brainstem at latencies only fractionally longer than vestibular ones and it could be important in detecting a fall and triggering motor responses

Other Conclusions Include:
a. EMG responses in younger normal subjects occurred at: sternomastoid 54 ms, abdominals 69 ms, quadriceps 78 ms, deltoid 80 ms, and tibialis anterior 85 ms. This pattern of muscle activation, which is not a simple ostrocaudal progression, may be temporally/spatially organized in the startle brainstem centers.
b. Voluntary tibialis EMG activation was earlier and stronger in response to a startling stimulus (fall) than in response to a nonstartling stimulus (sound). This suggests that the startle response can be regarded as a reticular mechanism enhancing motor responsiveness.
c. Elderly subjects showed similar activation sequences but delayed by about 20 ms. This delay is more than that can be accounted for by slowing of central and peripheral motor conduction, therefore suggesting age dependent delay in central processing.
d. Avestibular patients had normal latencies indicating that the free fall startle can be elicited by non-vestibular inputs
e. Latencies in patients with idiopathic Parkinsons disease were normal whereas responses were earlier in patients with multiple system atrophy (MSA) and delayed or absent in patients with Steele-Richardson-Olszewski (SRO) syndrome. The findings in this patient group suggest:
   i. Lack of dopaminergic influence on the timing of the startle response
   ii. Concurrent cerebellar involvement in MSA may cause startle disinhibition.
   iii. Extensive reticular damage in SRO severely interferes with the response to free-fall.

### (e) Experiment Protocol

Based on the results of Bisdorffs shown above, the following protocol for experimentation was developed

(i) Test A

Two rounds of testing were performed. For the first round, test A, six healthy volunteers (3 male, 3 female) between the ages of 21 and 35 years old were recruited. Five wireless sensors S1, S2, S3, S4 and S5 were positioned in five different places as shown in FIG. 2.1. Each person performed different fall types including forward, backward, and sideways, falling while transitioning from chair to standing position. Non-fall data was also recorded that included walking and bending.

(ii) Test B

For the second round of experiments ten healthy volunteers (5 male, 5 female) between ages of 21 and 40 years old were recruited. Similar to the first experimental set, the subjects performed the same fall types but this time the data was collected only from sensors S1, S2, and S3 for simplicity and high noise in the activity of lower extremities. The network camera was also used in test B to record all the activities, falls and no falls, of every subject.

The sensors used for Test B set of experiments are from Freescale Semiconductors, called the ZSTAR3 model, shown in FIG. 2.2. The ZSTAR3 has a MMA7361LT low power capacitive accelerometer on it. Three ZSTAR3 sensors are placed on each of the patients' bodies, and the data is acquired using a wireless USB stick that is also available from Freescale Semiconductors. The wireless communication is done at 2.4 GHz Radio Frequency. A ZSTAR3 tri-axial accelerometer sensor has a selectable data rate of 30, 60 or 120 Hz. The wireless range of these sensors is up to 20 meters. It consumes 1.8 to 3.9 mA of current during normal mode of operation. A coin sized CR2032 3V battery powers the sensors.

During the data acquisition from accelerometer sensors, all the falls and non-fall events are recorded using an IP camera to have a log of fall time so that fall time data can be used while processing the data for thresholds using decision trees. The IP camera used in this project is an IQinVision Model IQEYE2803A4 camera. This IPcamera is set up using a File Transfer Protocol Server (FTP) and FTP client. The Filezilla software is set up in such a way that the camera data is transmitted to an external hard drive connected to a computer through Wi-Fi. The camera data is obtained in the form of sequential images with a time stamp on them

(ii) Test C

For the third round of experiments six healthy volunteers (4 male, 2 female) between ages of 21 and 40 years old were recruited. Similar to the second experimental setup, the subjects performed the same fall types but this time the industrial lift belt embodiment of the present invention was used. The details of this industrial lift belt embodiment are discussed below, and the embodiment is shown in FIGS. 2.3 and 2.4. FIGS. 2.3 and 2.4 illustrate the placement of sensors and the processing unit on the industrial lift belt embodiment.

The embodiment of FIGS. 2.3 and 2.4 are believed to have several features that make it superior to known sensor containing devices. For example, the embodiment of FIGS. 2.3 and 2.4 has an integrated wired sensor system because wireless sensors are prone to high power usage when compared to wired. Additionally, wireless sensors are more vulnerable to signal interference and they need to have individual power supplies.

Another advantage provided by the wired sensors of the present invention is that they are easier to carry than wireless sensors. Wireless sensors are not easy to carry, as they are not held together. Due to this reason they need to be calibrated each time when the patient uses a device having wireless sensors.

The new hardware prototype comes with a vest, to which all the three accelerometers are sewed and the processing unit is also attached to it. These accelerometers have long cables such that it can be adjusted on the vest for people of different heights. The processing unit handles the fall detection when thresholds are met and also it has a Bluetooth module to transmit the data to any Bluetooth enabled device wirelessly.

### 2. Hardware Design

This section provides the information about the hardware design of the present invention and the integration of sensors into an embedded system. The embedded system of the present invention uses an I2C communication between the micro controller and the sensors. The serial data from the micro controller is transmitted using a Bluetooth module.

### (a) The Embedded System

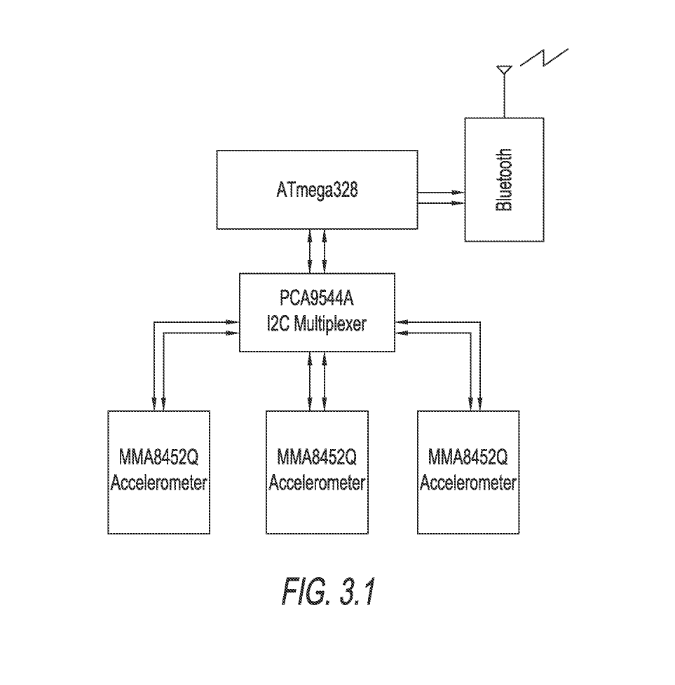

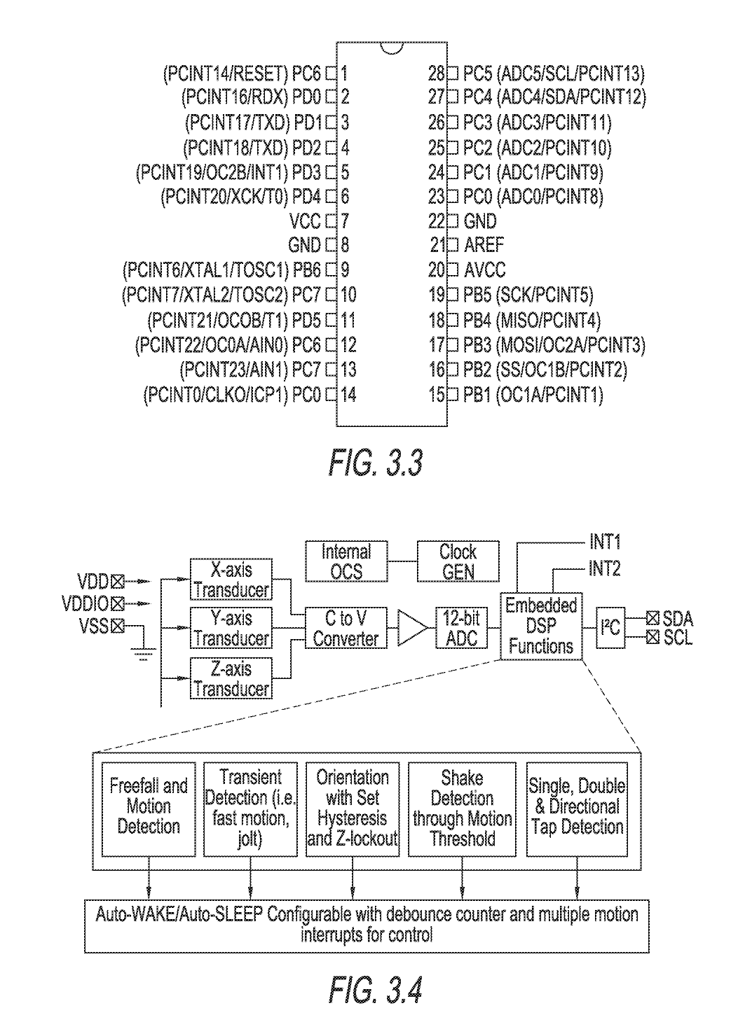

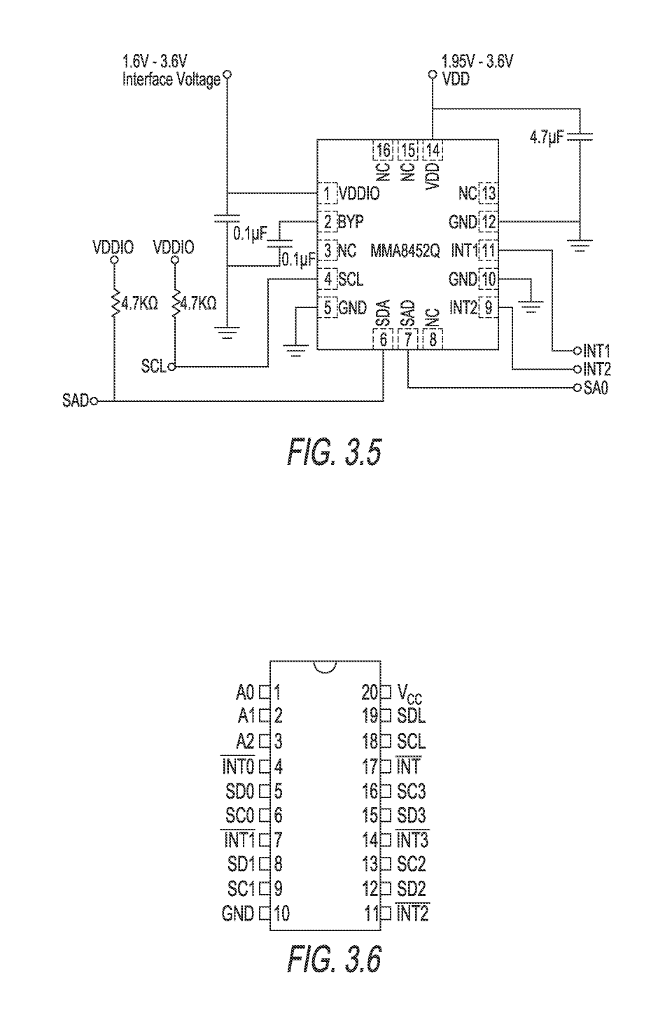

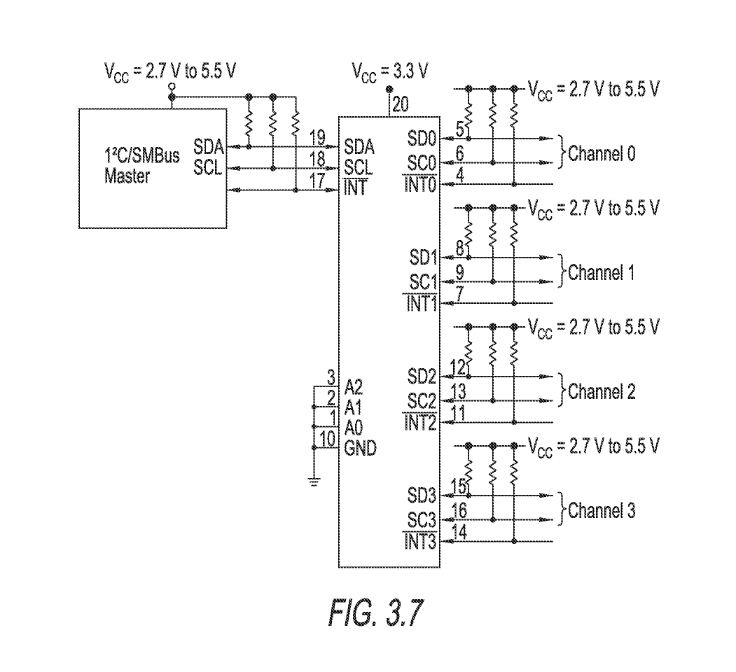

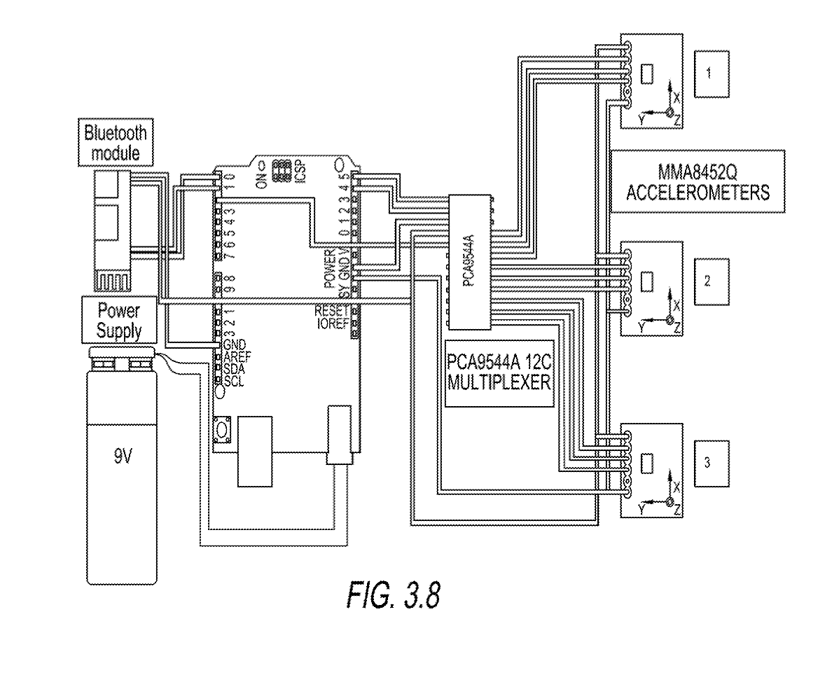

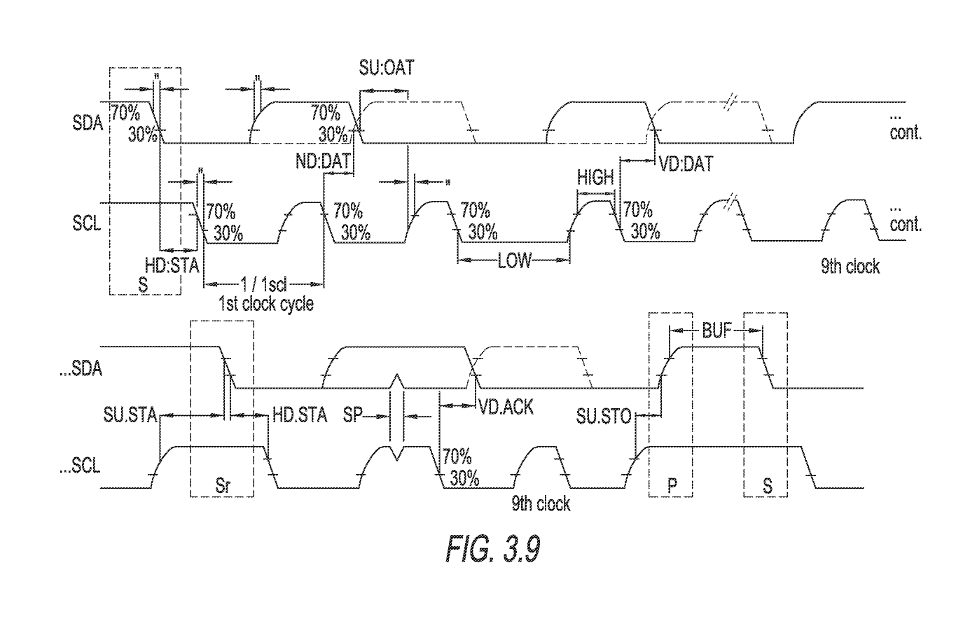

The integrated system consists of triaxial accelerometers, an I2C multiplexer, a micro controller and a Bluetooth module. FIG. 3.1 provides a basic block diagram layout of the integrated hardware and the types of communication between the components. The ATmega328 is the Major control unit, to which everything is integrated. The accelerometers are connected to this control unit through an I2C multiplexer such that the Microprocessor can distinguish between the three accelerometers, even though they have the same addresses. The information from the accelerometers is processed and the data is wirelessly transmitted to mobile devices or laptop using the Bluetooth module connected to it.

(1) Arduino

An Arduino UNO microcontroller unit used in this project is an open source hardware item. The Arduino microcontroller has an ATmega328 micro controller on it, and comes with a total of 14 digital input/output pins and 6 analog inputs. Out of the 14 digital input/output pins, 6 can be used as Pulse Width Modulation (PWM) outputs. It also has a 16 MHz ceramic oscillator onboard. Presented below is a block diagram of the Integrated sensor unit.

The ATmega328 has 32 KB Flash Memory, 2 KB SRAM and 1 KB EEPROM. The Arduino UNO board operating voltage is 5V and it has an onboard voltage regulator which can take up to a maximum of 20V from the supplied power jack. The board can be programmed using the USB port available and it can also be powered using the same port. The power jack provided can be used to run the Arduino when it is not used with the USB. There is an ICSP header for debugging and also a reset push button. In this project we are using the analog pins A4 and A5 pins to connect the SDA and SCK of the I2C multiplexer.

Digital pin D2 is used as logical low interrupt for the multiplexer. The Bluetooth module is connected to Rx and Tx pins of the Arduino so that the serial communication can be done wirelessly.

FIG. 3.3 shows the pin diagram of ATmega328

(2) MMA8452Q Accelerometer

The MMA8452Q (See FIG. 3.4) is a 3-Axis, smart, low-power, capacitive micromachined Digital Accelerometer from Freescale semiconductors with 12 bits of resolution. It is packed with two interrupt pins, which can be used to invoke the inbuilt flexible user programmable options and embedded functions. Those embedded interrupt functions allow for overall power savings relieving the host processor from continuously polling data.

The MMA8452Q has user selectable full scales of ±2 g/±4 g/±8 g with high-pass filtered data available for real-time applications. The communication is done using the I2C digital output interface. The MMA8452Q accelerometer has 42 configurable registers, which can be used based on the application. The acceleration data of the X, Y and Z-axes are stored as 2's complements of 12-bit numbers of the 6 registers from 0x01 to 0x06. Some of the features are motion freefall detection, tap and pulse detection, orientation, high pass filtering, Auto-sleep and wake up. The pin connection for the MMA8452Q are shown in FIG. 3.5

The Accelerometer is small enough for patients to wear. The MMA8452Q, when operating at 800 Hz consumes 165 μA current, making it a perfect choice for this application. FIG. 3.4 and FIG. 3.5 give the block and circuit diagrams that detail the internal architecture and pin connections of accelerometer.

(3) I2C Multiplexer for Accelerometers

The accelerometers used in design of this prototype are MMA8452Q from Freescale semiconductors. The data from the accelerometers are read using I2C communication. I2C communication is done based on the address of the slave units connected to the master unit. All the three accelerometers that are used in the design have the same address 0x2A.

A PCA9544A 4-channel I2C-bus multiplexer, a quad bidirectional-translating switch, is used to regulate the switching between the three accelerometers, where one SCL/SDA pair can be selected at a time. The PCA9544A provides four interrupt inputs and one open drain interrupt output. Whenever any device generates an interrupt, it is detected by the multiplexer and the interrupt output is driven low. Out of the four channels available, three were used to do the communication with the accelerometers. The multiplexer has a unique address of 0x70 while the SDA and SCL are connected to A4 and A5 of the Arduino. FIG. 3.6 illustrates the pin diagram and FIG. 3.7 illustrates the sample application of PCA9544A I2C multiplexer.

### (d) Bluetooth

The Bluetooth module used in this prototype is a factory configured serial data transmission board. It has Vcc, Tx, Rx, and ground pins of which the Tx of the Bluetooth is connected to Rx of Arduino, and the Rx of Bluetooth is connected to the Tx of Arduino in order to transfer the data wirelessly. The Bluetooth module is configured to 9600 Baud rate as a default setting. It can operate at a range of up to 30 ft and voltage range from 3.3 to 5

### (e) 3.2 Inter-Integrated Circuit Communication-I2C

Inter-Integrated Circuit is a bidirectional two-wire interface synchronous communication protocol. It requires two bus lines, Serial Data and Serial Clock

Each device connected to this bus is software addressable by a unique address. I2C bus is a multi-master bus where more than one integrated circuit is capable of initiating a data transfer can be connected to it, which allows masters to functions as transmitters or receivers. I2C communication is highly immune to noise, has wide supply voltage range that consumes very low current

In the present invention, the microprocessor acts as a master and the three triaxial accelerometers act as slaves. Both the bi-directional lines, SDA and SCL are connected to a positive supply voltage via 4.7KΩ pull up resistors. Data transfer rate on the I2C bus can range from 100 Kbits/s to 3.4 Mbits/s based on the application modes. A data START condition is observed when a HIGH to LOW transition on the SDA while SCL is HIGH. A LOW to HIGH transition on the SDA line while SCL is HIGH defines a STOP condition. These START and STOP conditions are always generated by the master. Once the START condition is initiated, the bus is considered as busy until a STOP condition is reached. FIG. 3.9 shows the signal integrity timing diagrams, including the START and STOP bits.

### (f) UART Communication

The Universal Asynchronous Receiver/Transmitter communication is used to transmit the data from three accelerometers to the mobile device using the Bluetooth module. Unlike I2C, UART is an asynchronous communication protocol (No clock required). Baud rate for the Bluetooth module used in this set up for the present invention is at 115200.

### (g) Hardware Programming

The wire and math libraries are included for the I2C communication and trigonometric functions respectively. Initially to begin the I2C communication with the accelerometers, the contents of 0x0D register is read using the readRegister user defined function. This readRegister function invokes the Wire.beginTransmission function of the Arduino library, which begins a transmission to the I2C slave device with the address 0x1D.

The Wire.write(0x0D) function writes the data from the accelerometer in response to a request from the ATmega328. The Wire.endTransmission(false) command is used not to send a STOP condition to the Wire.beginTransmission such that the I2C bus will not be released yet. This prevents another master device from transmitting between messages. This allows one master device to send multiple transmissions while in control.

Wire.request From (address, quantity) is used by the master to request bytes from a slave device. Wire.read( ) reads a byte that was transmitted from a slave device to a master after a call to requestFrom( ) was transmitted from a master to slave. The measured acceleration data of the MMA8452Q is stored in OUT X MSB, OUT X LSB, OUT Y MSB, OUT Y LSB, OUT Z MSB, and OUT Z LSB registers as 2 s complement 12-bit numbers. The most significant 8-bits of each axis are stored in OUT X (Y, Z) MSB.

The MMA8452Q has an internal ADC that can sample, convert and return the sensor data when requested. The 8-bit command transmission begins on the falling edge of SCL. The transaction on the I2C bus starts with a START condition signal. After START condition has been transmitted by the master (ATmega328), the I2C bus is considered as busy.

The next byte of data transmitted after START contains the slave address in the first 7 bits, and the eighth bit is reserved to indicate whether the master is receiving data or transmitting data.

The MMA8452Q is set to operate at 800 Hz (Maximum available) such that it can transmit 84 samples per second when 115200 baud rate is used. Signal features SVM, SMA and Tilt angle are calculated and the thresholds are set such that whenever there is a fall occurrence, the LED pin connected to the 12th pin of Arduino is turned on and the event is logged on the PC.

The Arduino UNO board is programmed using the Arduino software and the code is disclosed in the above referenced provisional application that is fully incorporated herein by reference. FIG. 3.8 schematically illustrates the circuitry of the power source, Bluetooth module, processor and accelerometer.

FIG. 3.10 and FIG. 3.1 show a respective front and back view of proposed hardware prototype, green blocks represent the accelerometers and the red represents the processing unit

### 4. Pattern Recognition and Data Acquisition

In the present invention, the two preprocessing steps are used. The first step is median filtering and the second step is low pass filtering. The low pass signal filtering is considered as an estimation of the gravitational acceleration (GA), and the median filtering is an estimation of the body acceleration (BA).

### (a) Feature Extraction Indices SVM, SMA, Tilt Angle

We used the second algorithm presented by Karatonis et al. in D. Karantonis, M. Narayanan, M. Mathie, N. Lovell, and B. Celler, “Implementation of a real-time human movement classifier using a triaxial accelerometer for ambulatory monitoring,” Information Technology in Biomedicine, IEEE Transactions on, vol. 10, no. 1, pp. 156-167, January

The algorithm is based on the assumption that a fall is a signal of extreme impact. The degree of movement intensity is known as signal vector magnitude (SVM) and it is derived from the BA component as follows:
222

SVM[i]=XBA[i]+y_{nA}[i]+zBA[i] (4.1)

where xBA[i] is the i^{th} sample of the BA component along the axis samples (similarly for yBA[i] and ZBA [i]). Comparing the SVM with a threshold helps determine the fall event. In order to measure the intensity of the activity and distinguish between rest and movement, the signal magnitude area (SMA) is calculated. SMA is the sum of the integrals of the three acceleration signal magnitudes and it is also calculated using the BA component as shown:

j=i−T
1

SMA[i]=(|XBA[j]|+|yBA[j]|+|zBA[j]|) (4.2)
T
   j=i

where XBA[i], yBA[i], and zBA[i] are the BA components of the x, y, and z axis signals and T is the sampling period. Using the GA component of the signal helps determine the postural orientation of the subject wearing the accelerometers. The derivation of tilt angle can be achieved using the GA component along the z axis as
zGA[i]

Φ[i]=cos⁻¹ (4.3)
222

xGA[i]+y_{GA}[i]+zGA[i]

where xGA[i] is the i^{th} sample of the GA component along the axis samples (similarly for yGA[i] and zGA[i])

### (b) Threshold Information

Using data collected after Test A and Test B described above and video recordings of the fall, image processing techniques were used to classify the accelerometer data as fall and no fall events based on the body inclination. The images were processed in order to determine the moment in which body inclination was between 15 and 60 degrees with respect to the vertical axis. Using that moment in time where the image reached the range, the accelerometer data was then classified as fall or no fall (0 for no fall and 1 for fall). Matrices containing the classification array of zeros and ones, and arrays of SMA values, SVM, and Tilt angles for the three sensors were created and used to find thresholds using a decision tree model.

### (c) Decision Tree Model

Decision trees are pattern recognition tools that provide weighted solutions to a classification problem with output classes such as fall/no fall in our case. Decision Trees are constructed from training data sets in which each data point contains an input vector along with a target value. The target value, either a 1 or a 0, represents the class to which the data belongs. The software Rattle, a sub-package of R was used for the purpose of training and calculating the fall/no fall threshold values.

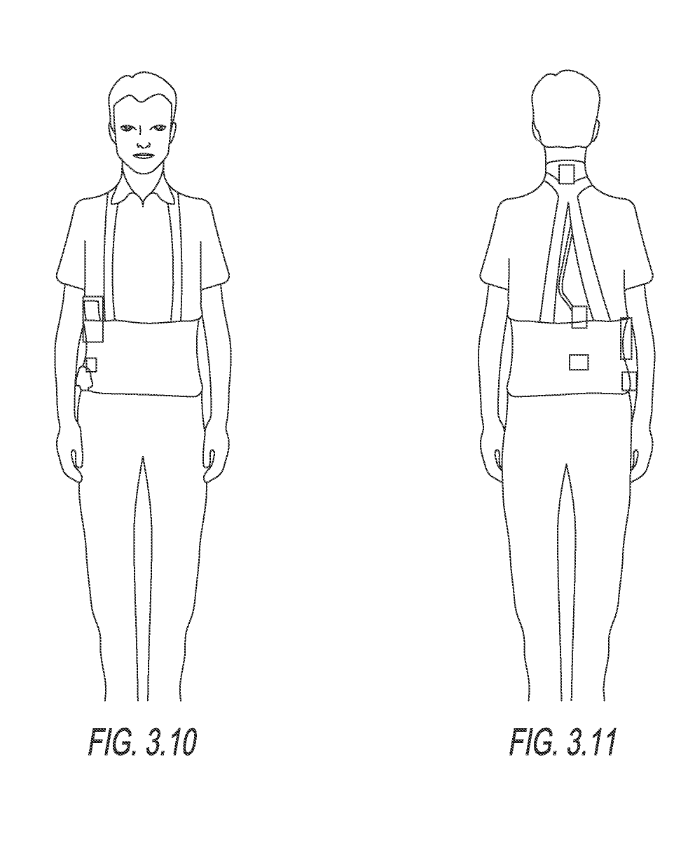

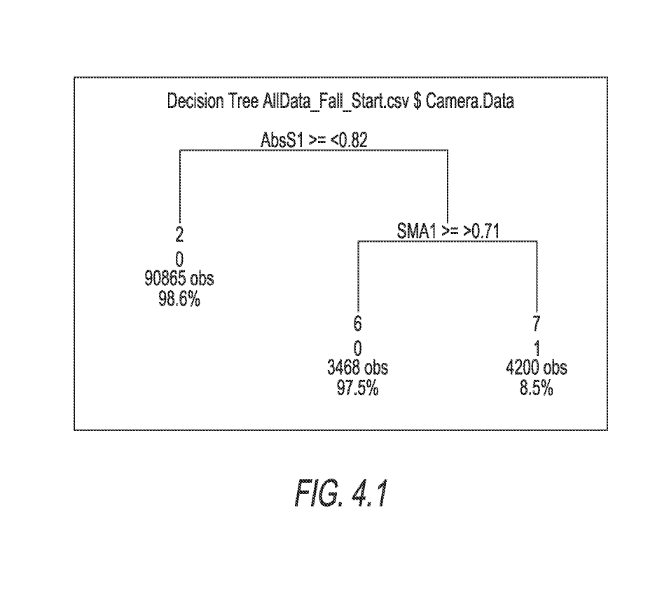

These thresholds are later coded using if-then statements and later stated on unseen data points as the prediction is compared with true classes. FIG. 4.1 shows a sample DT as a flowchart with rules. Two parameters in Rattle are adjusted to modify the output: complexity cost and loss matrix. The complexity cost is a number between 0 and 0.0001 that adjusts the size of the tree. The larger the complexity costs the simple decision tree containing fewer nodes. The loss matrix is a comparative misclassification cost used to make fall or no fall class almost pure

### (d) Falls and Non Falls Setups

Using the new hardware prototype of the integrated sensor system, data was collected from 6 subjects (4 male and 2 female). The age, height and weight of all subjects were documented. All the six subjects were asked to wear the vest, to which the sensors and the processing unit were attached. Seven different activities, which imitate both falls and non-falls, are asked to perform
a. Frontal fall: Subjects were asked to take two laps of normal walking around the mattress and to imitate a frontal fall on the mattress.
b. Side fall: Subjects were asked to take one lap and take a side fall on the mattress.
c. Back fall: This fall was taken without walking, but asked to fall down backwards.
d. Chair fall: Before the data acquisition, the subject will be sitting in a chair and asked to imitate a chair fall while standing up, and the data is collected.
e. Sit normal in a chair: This involves the subject sitting in a chair normally.
f. Sit suddenly in a chair: In this activity, the subject is asked to sit suddenly and it should be considered as a non-fall by the detection unit.
g. Tripping: Subjects are asked to walk normally for some time, then imitate a trip near a window but prevent themselves from falling. This should be detected as a no fall by the hardware unit.
h. Lay normally on a bed: Subjects were asked to walk around and lay normally on a bed.
i. Lay suddenly on a bed: This is similar to normal laying but the subject will be doing it with a sudden movement.

Of these seven activities, the first four (activities a-d) are the real falls and the latter five (e-i) are non-falls. Accuracy is determined based on the true positives, true negatives, false positives and false negatives of the fall detection. The following figures illustrate some of the fall types and also give an idea of the test setup.

### (e) Data Acquisition Using Bluetooth Enabled Laptop

The serial data transmitted by Bluetooth module connected to the processing unit can be saved on any computer that has Bluetooth capability. The pairing password for the Bluetooth module is 1234 by default. The baud rate was set to 9600 as factory default. As we need to transfer our serial data at 115200 baud rate, it can changed by sending some AT commands to it. AT+BAUD8 command will change the baud rate from 9600 to 115200. Serial data from the Bluetooth can be saved on a computer in command separated values file version (csv) using MATLAB as shown in FIG. 4.6.

The Bluetooth device can be identified and used to save data using the command: s=serial(‘/dev/tty.BTUART-DevB’); for Mac s=serial(‘COM4’); for Windows set(s, ‘BaudRate’, 115200); is used to set the baud rate of the port, datestr(now,‘HH,MM,SS,FFF’); is used to store the data with a time stamp in Hours: minutes: seconds: milliseconds format.

The falls are detected in real time using the thresholds set on the signal features. During the experimentation process, the data is transmitted in real time to a Bluetooth enabled device to verify the accuracy of hardware prototype. In the aforementioned provisional application, several different types of falls are illustrated. FIGS. 4.2 to 4.5 and FIG. 4.7 of the provisional illustrate a frontal fall graphically with the SVM from 3 accelerometers. These figures from the provisional are fully incorporated herein by reference.

### 5. Results

Three different tests were performed. The first two tests used two different resulting data sets from Test A and Test B described in the experimental protocol section and generated fall detection simulations using MATLAB. Prior to testing for fall detection accuracy, it was necessary to test the resulting decision tree thresholds on a series of consecutive samples of the fall data after the first time the threshold was met.

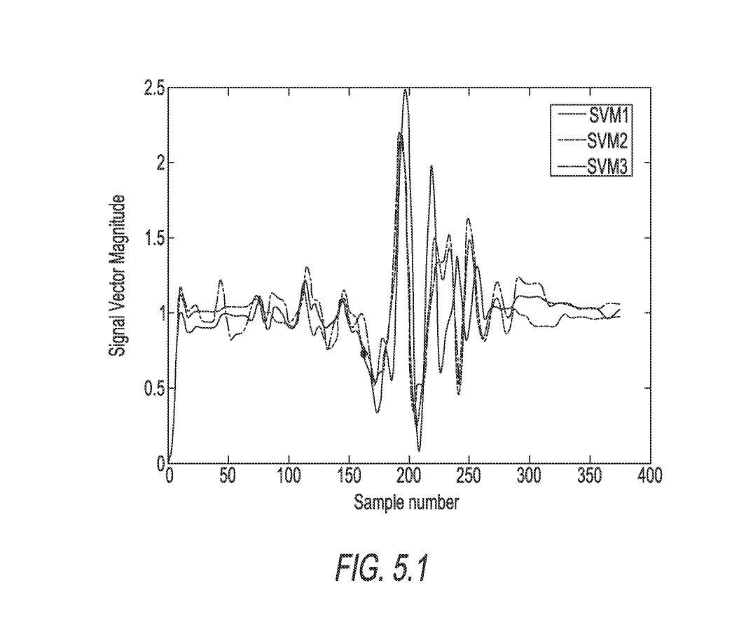

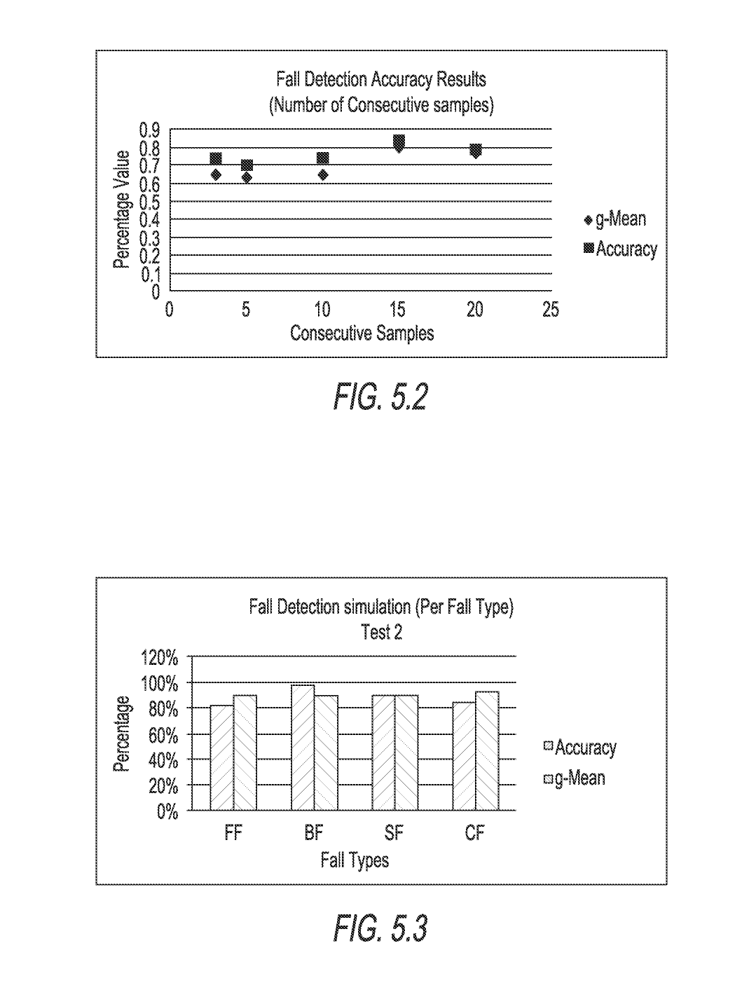

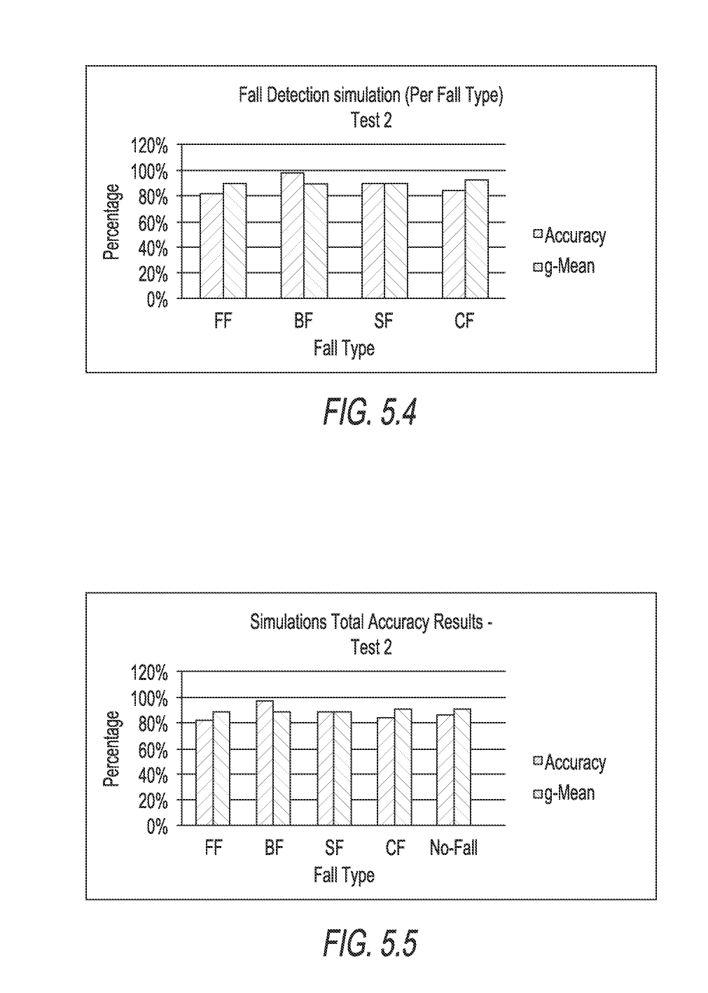

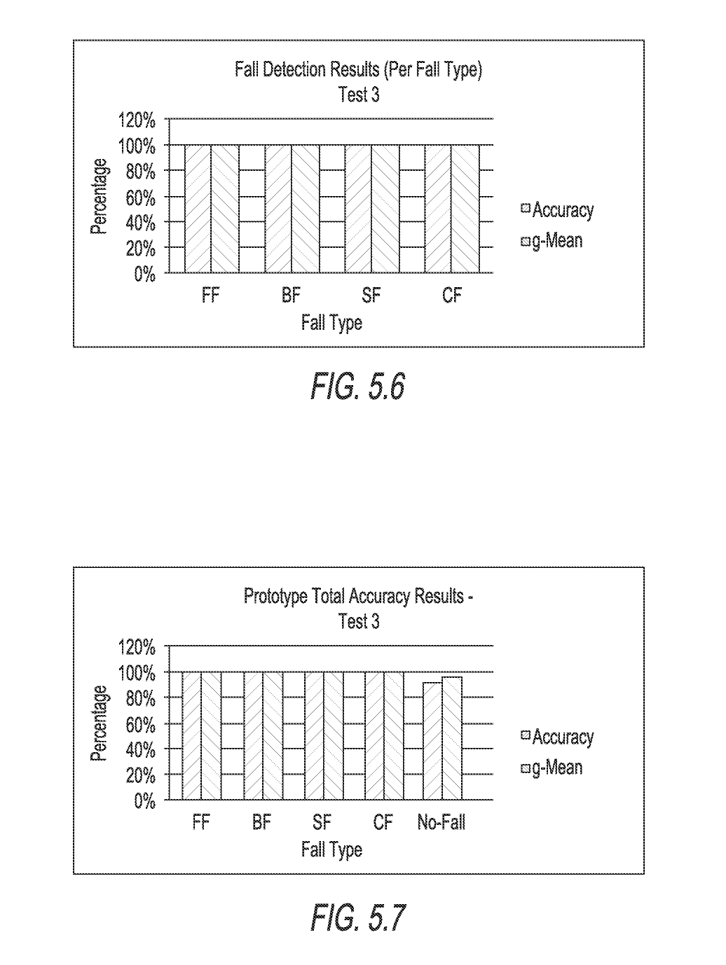

FIG. 5.2 shows accuracy results of fall detection using 5 to 25 consecutive samples after the first time the threshold is met. It was concluded that testing the threshold on 15 consecutive samples was the best option with about 86% accuracy. Once the test range was determined, the following tests were performed:

### (a) Test One

The data set generated using collected data in Test B was enrolled in the decision tree software. The output thresholds were then tested on data generated using Test B data. FIG. 5.1 shows a fall detection simulation output. The green dot shows where the algorithm detected the fall. Fall detection classification was done as follows:
a. A true positive occurs when a green dot lied before the lower most point of the plot as shown in FIG. 5.1. We know that the fall trajectory occurs before the lowest peak based on the video images. In this case a fall was correctly identified. A fall positive occurs when a green dot appears in no fall data sets (i.e. tripping, sudden sitting). In this case no-fall data was incorrectly classified.
b. A true negative occurs when a no-fall data set is correctly classified or when a green dot does not appear in no-fall data.
c. A false negative occurs when a fall data set is incorrectly classified or when a green dot does not appear in a fall data set.

Fall detection accuracy results of Test One are represented in FIG. 5.3

### (b) Test Two

The data set generated using collected data in Test B was enrolled in the decision tree software. The output thresholds were then tested on data generated using Test A data. Fall detection accuracy results for falls only of Test Two are represented in FIG. 5.4. FIG. 5.5 shows test results using thresholds on the data collected for a total of sixteen subjects

### (b) Test Three

In test three, the hardware prototype was tested. Fall detection was performed by the microprocessing unit in real time. Classification was recorded based on whether the LED light went on during falls or other no-fall events or movements. Using the output threshold values of the decision tree models and programming them in the microprocessing unit, the prototype was tested in several frontal falls and data was collected.

However, falls were not being detected under those threshold conditions. Using the new data of frontal falls generated by the hardware prototype, and observing the threshold values generated by the decision tree model, an informed selection of thresholds was performed as follows:

For every fall, one SMA and one SVM, value for the three sensors were manually chosen from the range where fall happens (right before the lowest acceleration value). For simplicity and because high fluctuation of Tilt angle values, it was decided to only select SMA and SVM values.

Out of all the falls, the lowest values of SMA, SVM, were selected. It was determined to select the lowest values because it would guarantee a closer threshold to the beginning of the fall.

Manual thresholds were reprogrammed in the microprocessor.

FIGS. 5.6 and 5.7 show the results of test three.

### 6. Conclusion and Future Work

### (a) Conclusion

This application addresses how an integrated sensor system was designed for early fall detection in elders. In an initial phase, a deep understanding of neuroscience and the relationship between brain activity and fall events was developed through research. Then, a wireless sensor unit from Freescale (ZSTAR3) was used for data acquisition.

For the initial set of experiments, a total of sixteen subjects performed seven different kinds of falls as well as no-fall activities. Data from the wireless triaxial accelerometers was used to calculate signal features like Signal Vector Magnitude, and Signal Magnitude Area, and Tilt Angle.

These features were tested and simulated with MATLAB software, against each fall data set to determine thresholds, which were obtained using decision trees. Once the data was processed, a decision tree model was used to determine fall detection thresholds. A hardware prototype was then developed. This hardware features low power, high-speed sensors and processing units.

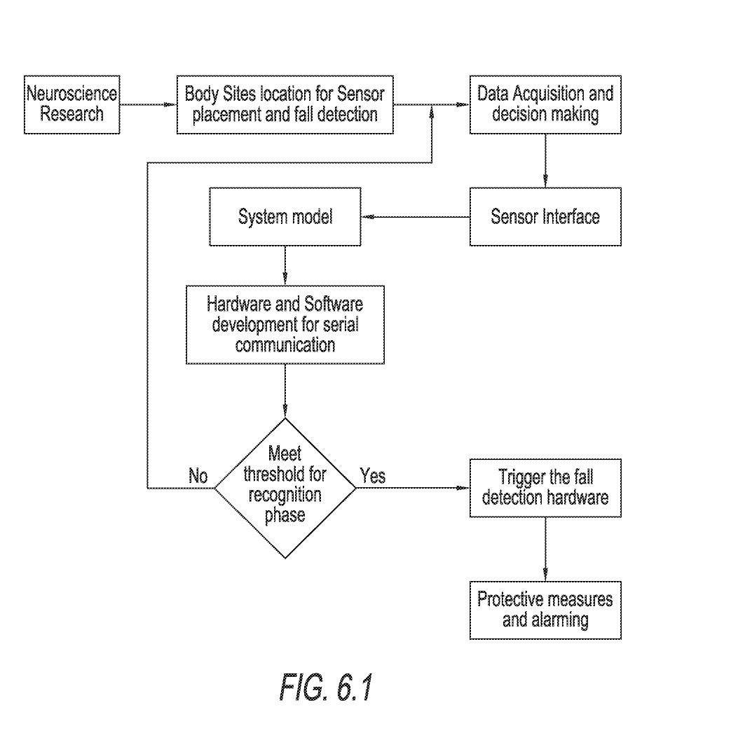

The prototype, in which the calculated thresholds were programmed, was tested with a final set of experiments in which six volunteers were asked to imitate seven different kinds of falls while wearing the hardware prototype. Once again, the test included falls and non-falls. Accuracy was measured separately for total number of actual falls and total number of activities (which include both falls and non-falls). The new hardware prototype had an accuracy of 100% in detecting fall events and 95.55% accuracy in the case where all the fall and non-fall events are included. FIG. 6.1 shows the closed loop functioning diagram of the project.

### (b) Additional Features and Options

This application discloses an efficient working prototype of a fall detection unit with deployable airbags. It is believed that the sensor system can be improved to lessen the occurrence of false positives by adding a gyroscope to classify both angular velocity and body position. Observing 84 samples per second at the receiving end of the Bluetooth has brought sufficient resolution to detect the fall event. Employing other sampling rates may help to optimize noise, calculation time, and robustness to achieve better real time application.

The sensor and controller system is integrated into the vest of the present invention, to make use of the deployable air bags that are deployed using portable pressurized air cylinders to prevent hip and neck fractures during a fall event. Research in determining the angle of impact will be helpful in deploying airbags in an intelligent way and it should be classified based on factors like height, weight and age.

The system can also include a communication system that uses a cellphone application that integrates emergency services to assist people who have experienced a fall. The system can also include a log feature where data can be saved and used for further classification and specification of activities.

As with many devices that include sensors and compressed air sources, it may be useful to establish a reliable useful life span for the device. Such a life span could be used to determine an expiration date for the device to ensure that the device operated reliably and when needed, and did not fail due to age related reasons. Alternatively, the controller or an outside controller could be programmed to enable the user to conduct tests of the device at predetermined time intervals to ensure that the device was still functioning properly. Similarly, the device could include an alarm, similar to a smoke detector, the would send an audio or light related signal to the user to inform the user that either (1) the device was in need of testing; or (2) that the power source for the device (e.g. battery) was running low on charge and was in need of being replaced or recharged.

What is claimed is:

1. A protective clothing article including a wearable member placeable over the torso of a user, comprising
an upper portion for engaging the user's shoulders
a lower portion disposed, when worn by the user, at a vertical position generally similar to the pelvis of the user,
at least two deployable airbags disposed on the lower portion of the clothing article at a vertical position generally similar to the pelvis of a user,
a compressed air source for injecting air into the air bags upon deployment of the air bags to inflate the airbags,
at least two sensors capable of detecting and sensing information relating to the direction and velocity of movement of the user, and
a controller in communication with the sensor for processing sensed information from the sensor and processing said sensed information to determine whether a fall event is imminent and, upon determining that such a fall event is imminent, sending a signal to the compressed air source to inflate and thereby deploy at least one of the two deployable airbags.

2. The protective clothing article wherein the at least two sensors include a first sensor, a second sensor and a third sensor.

3. The protective clothing article wherein the useable member includes a back portion placeable adjacent to the user's back, and wherein the first sensor is placed on the back portion to be disposed adjacent to the user's back.

4. The protective clothing article of claim 3 wherein the second sensor is disposed on the lower portion of the protective clothing article, and is positioned to be placeable adjacent a side of the user.

5. The protective clothing article of claim 4 wherein the third sensor is placed on the back portion of the protective clothing article and is positioned generally above the first sensor.

6. The protective clothing article of claim 5 wherein the first, second and third sensors comprise accelerometers.

7. The protective clothing article of claim 1 wherein the at least two sensors comprise accelerometers.
